(19) United States
(12) Patent Application Publication
(10) Pub. No.: US 2022/0226136 A1
(43) Pub. Date: Jul. 21, 2022

(54) DEVICES AND METHODS FOR TINNITUS TREATMENT

(71) Applicant: Sonorous, NV, Lake Forest, CA (US)

(72) Inventor: Charles E. Stout, Moreno Valley, CA (US)

(21) Appl. No.: 17/667,425

(22) Filed: Feb. 8, 2022

Related U.S. Application Data
(63) Continuation-in-part of application No. 17/137,324, filed on Dec. 29, 2020.

(51) Int. Cl. A61F 2/966

(52) U.S. Cl. CPC A61F 2/966; A61F 2210/0004; A61F 2250/0098

(57) ABSTRACT

Detachable stents delivered to the transverse, straight or superior sagittal sinus used to address tinnitus and related venous abnormalities including stenosis, jugular bulb and arachnoid granulation, inter alia.

### CROSS-REFERENCE TO RELATED APPLICATIONS

The present application is a continuation of U.S. Ser. No. 17/137,342, filed Dec. 29, 2020, which claims full priority of U.S. Ser. No. 62/957,090, filed Jan. 3, 2020. All Paris convention rights are expressly reserved.

### BACKGROUND OF THE INVENTION

The present inventions relate to endovascular treatment of venous abnormalities including stenosis, jugular bulb and arachnoid granulation, inter alia.

### OBJECTS AND SUMMARY OF THE INVENTION

Briefly stated, detachable stents delivered to the straight, transverse, or superior sagittal sinus, inter alia, address tinnitus and related venous abnormalities.

According to embodiments, detachment of stent may be done electrolytically, mechanically or by any other known methods.

According to embodiments, flow diverter/stents may be mesh or laser cut and has an inherent density resulting in flow diversion.

According to embodiments, flow diverter/stents may have properties of trackability and softness to enable, with normal and known access, advancement to the straight or superior sagittal sinus.

### BRIEF DESCRIPTION OF THE DRAWINGS

Various preferred embodiments are described herein with references to the drawings in which merely illustrative views are offered for consideration, whereby:

FIG. 1 shows a pusher wire and detachable stent, along with detachment zone associated therewith;

FIG. 2 shows steps for the procedure to resolve tinnitus (sometimes done with patient awake); and

FIG. 3 shows the stent of FIG. 1, detached and/or re-sheathed, per the claimed procedure, as emplaced with detailed view of pressure gradient inducement.

Corresponding reference characters indicate corresponding components throughout the several views of the drawings. Skilled artisans will appreciate that elements in the FIGURES are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION OF THE INVENTIONS

The present inventor has developed a procedure whereby patients may be treated for tinnitus, often while awake, to verify resolution of the same.

The present inventor likewise addresses stenosis, jugular bulb, arachnoid granulation and other venous abnormality found by venogram.

Expressly incorporated herein by reference, as if fully set forth herein in their entirety are U.S. Pat. Nos. 9,808,359 and 10,398,877. These Letters Patent demonstrate those materials and methods known to those skilled in the art for neurovascular procedures, materials and processes, and vascular access to both the peripheral vascular system and major arterial and venous sites for said procedures, along with the instant teachings. It is respectfully proposed that progress in the useful arts has been made by discovery of a procedure to resolve tinnitus, using materials and methods for these references.

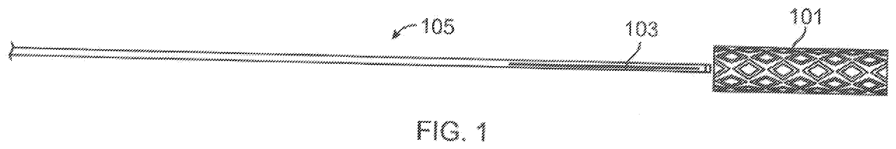

Referring now to FIG. 1, those skilled in the art understand that using materials and methods from U.S. Pat. No. 9,808,359 (Ferrera) a detachable stent 101 or flow diverter attaches to a pusher wire 105, and separates at detachment zone 103. Access is achieved per the Seldinger technique and as detailed in U.S. Pat. No. 10,398,877 (Vitullo).

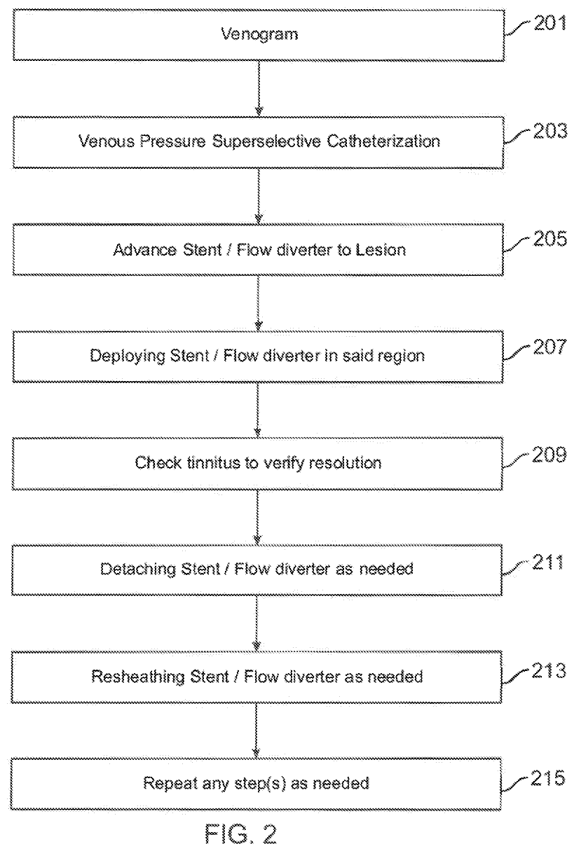

Referring now to FIG. 2, a schematic or cartooned flow chart shows the steps of the instant procedure, namely: Generating at least a venogram 201; taking venous pressure 203 via super selective catheterization; advancing stent/flow diverter 205 to the subject venous lesion; deploying stent/flow diverter 207 in said lesion; Checking Tinnitus or pressure 209 to verify resolution; Detaching stent/flow diverter 211; Resheathing stent/flow diverter 213; and repeating as needed 215.

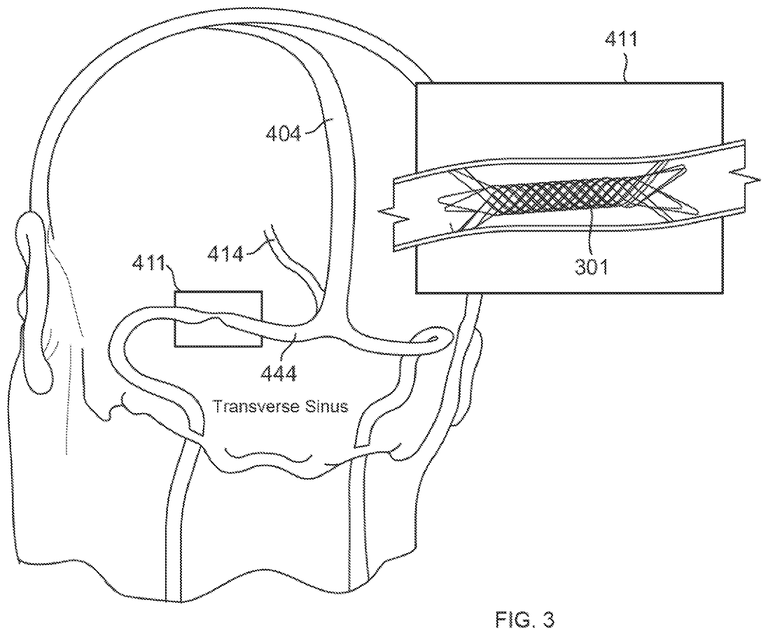

FIG. 3 shows stent/flow diverter 301 detached from pusher wire 305 at detachment zone 303 (not shown), transverse sinus 444, shows a pinched aspect of patient's left transverse sinus branch, extending distal from tocula—or bifurcation. Artisans understand that any vascular abnormalities causing pressure gradients may be stented, anywhere from superior sagittal sinus 404 to straight sinus 414, or anywhere accessible within the brain, to normalize blood flow patterns, thus resolving perceived and actual tinnitus, pulsatile or otherwise characterized. Conscious sedation supports immediate feedback on treatment. Referring now to all the case there is disclosed a set of procedures that address, attenuate, mitigate or otherwise eliminate tinnitus. Further encompassed within this case are the specific devices, which embody the stents/flow diverters of the present inventions which have a density to function as flow diverters which being pliable, conformable and trackable as stents ensure they conform with Nitinols and alloys and hybrids which can be woven into open celled configurations or laser cut and tubes, rigid enough to be directed, but having those specific characteristics as known to work here.

It is likewise claimed that any system components or elements herein denoted for the first time are proprietary and as disclosed within this application and the appendix of its provisional from which priority is claimed specifically the stent/flow diverters and any other surgical appliances and devices, including coatings, coverings, drug-elution, protein elution and capture and the like antimicrobial means.

Further non excluded are the electronics covering all data generated through these procedures and any specialized computing means embodying the same including personalized digital assistant and AI means incorporated in the same.

Artisans understand that detachment of stent may be electrolytic, mechanical or other, and that the stent may be mesh or laser cut. Likewise, the stent may have density resulting in flow diversion. Finally, stent is trackable, and has a stiffness/softness ratio allowing it to be advanced to straight or superior sagittal sinus.

According to this genus any endovascular approach allows to access the superior sagittal sinus (superior longitudinal sinus) may be used. Then any variation on these steps works: using a venogram to verify at least one venous abnormality from the group consisting essentially of stenosis, jugular bulb issues, arachnoid granulation, and the like malformations and/or structural deformations of the vasculature; optionally employing super selective catheterization to measure venous pressures; advancing at least a stent to a subject venous lesion or more than one; deploying the at least a stent for optimal location within the lesion(s); checking the pressure or tinnitus level (with or without conscious presence of the patient) for the verification of the desired outcome or resolution; and, detaching the at least a stent or re-sheathing the same in the event of not achievement of the desired outcome.

Those skilled understand that the stent needed for this approach have to balance stiffness, radial force and softness. Such stents have been developed and are subject to co-pending IP. Expressly referenced is U.S. Ser. No. 63/060,040 (Aug. 3, 2020).

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment,” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention, and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or I ntel Xeon E7-2820).

An I/O interface may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antennae, and a network interface device (e.g., a network interface card (NIC), Wi-Fi card, cellular modem, data jack, Ethernet port, modem Jack, HDMI port, mini-HDMI port, USB port), touchscreen (e.g., CRT, LCD, LED, AMOLED, Super AMOLED), pointing device, trackpad light (e.g., LED), light/image projection device, or a combination thereof.

Memory according to the invention refers to a non-transitory memory, which is provided by one or more tangible devices which preferably include one or more machine readable medium on which is stored one or more sets of instructions (e.g., software) embodying any one or more of the methodologies or functions described herein. The software may also reside, completely or at least partially, within the main memory processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof In certain embodiments, a device of the itivemion includes a tangible, non-transitory computer readable medium for memory. Exemplary devices for use as memory include semiconductor memory devices, (e.g., EPROM, EEPROM, solid state drive (SSD), and flash memory devices, (e.g., SD, micro SD, SDXC, SDIO, SDHC cards); magnetic disks, (e.g., internal hard disks or removable disks); and optical disks (e.g., CD and DVD disks).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An improved procedure to alter pressure gradients, in a stented vessel/vein/sinus system, which comprises, in combination:
accessing the cerebral vasculature;
measuring venous pressure with at least one of a pressure wire and alternate turbulent flow pattern locator;
delivering a sterile single use soft and compliant stent/flow diverter; and,
releasing and/or re-sheathing said stent/flow diverter by command of the operator.

2. The improved procedure to alter pressure gradients of claim 1, whereby the step of releasing further comprises:
checking pressure in stented vessel/vein/sinus system.

3. The improved procedure to alter pressure gradients of claim 2, further comprising:
confirming resolution of the pressure gradient.

4. The improved procedure to alter pressure gradients of claim 3, further comprising:
active sedation, meaning patients may be treated with general, but not wakeful consciousness-impacting therapies.

5. The improved procedure to alter pressure gradients of claim 4, which may be repeated, subject to pressure gradient measurements communicated from the pressure wire.

6. The improved procedure to alter pressure gradients of claim 5, used to address via in situ diagnosis and treatment with immediate feedback on treatment success or no-effect.

7. The stented vessel/vein/sinus system addressed being at least one targeted zone of interest from the group of:
areas impacted by vascular causes or arterial or venous vascular pathologies (dual arteriovenous fistula [dAVF], arteriovenous malformation [AVM], aneurysm, ICA stenosis/dissection, congenital vascular variants, transverse sinus stenosis), or increased cardiac output.

8. The stented vessel/vein/sinus system addressed further comprising:
at least one non-vascular etiology driven type of pulsatile tinnitus from the group of neoplasm like paranglioma or osseous pathology, idiopathic intracranial hypertension, and systemic disorders such as anemia.

9. The system of claim 7, wherein it is used to address venous stenosis.

10. The system of claim 9, wherein the patient remains awake to verify resolution.

11. A system for addressing venous abnormalities, which comprises, in combination: a pusher wire attached to a detachable stent/flow diverter; the detachable stent/flower diverter being trackable and having adequate softness to advance to the straight or superior sagittal sinus.

12. The system of claim 11, wherein the stent/flow diverter is mesh.

13. The system of claim 11, wherein the stent/flow diverter is laser cut.

14. The system of claim 11, wherein the detachable stent has the density to function as a flow diverter.

15. The system of claim 14, further comprising microcatheters for introducing the system into an access port.

16. The system of claim 15, further comprising radiopacity for visualization.

17. The system of claim 16, further comprising electromechanical detachment.

18. The system of claim 17, further comprising mechanical detachment.

19. The system of claim 18, further comprising a bio-resorbable stent.

20. The system of claim 19, further comprising a drug-eluting, coated or covered stent.
